(19) United States
(12) Patent Application Publication
(10) Pub. No.: US 2010/0279344 A1
(43) Pub. Date: Nov. 4, 2010

(54) Integrated cytokine production system

(75) Inventor: Victor Smit, Almere (NL)

Correspondence Address: FITCH, EVEN, TABIN & FLANNERY, P. O. BOX 18415, WASHINGTON, DC, 20036, US

(73) Assignee: Stiching Pharma Park IP, Almere (NL)

(21) Appl. No.: 12/000,470

(22) Filed: Dec. 13, 2007

Related U.S. Application Data
(60) Provisional application No. 60/874,487, filed on Dec. 13, 2006.

(51) Int. Cl. C12P 21/00

(52) U.S. Cl. 435/69.1

(57) ABSTRACT

A multi-stage method for producing a cytokine involves expressing the cytokine in an expression system, preferably a Bacillus subtilis strain from which at least 7, and preferably at least 8, extra-cellular proteases have been eliminated that additionally provides a significantly increased expression and secretion of the desired cytokine, subsequently purifying the cytokine under mild conditions that are as natural as possible and that also preferably encompass mild materials, recovering and, optionally, isolating the cytokines that were excreted from the expression system (bacteria).

### RELATED APPLICATIONS

This is a utility application (“complete” application) that claims the priority and filing date benefit of U.S. Provisional Application No. 60/874,487, filed Dec. 13, 2006, the complete disclosure of which is incorporated herein by reference.

### FIELD OF THE INVENTION

The present invention relates to a method for producing cytokines and cytokine derived biomolecules in large amounts with a high degree of purity, and in a highly natural manner. More particularly, production takes advantage of intrinsic properties of the cytokines that also enable their function in the human body namely: 1) under natural circumstances cytokines are excreted by cells; and 2) in their natural state cytokines are just a little (slightly) but not too hydrophilic as they would otherwise stick to any cell membrane; and are just a little (slightly) but not too heavily charged as they would otherwise be neutralized and excreted by the human body mechanisms too rapidly.

### BACKGROUND OF THE INVENTION

The global market for biopharmaceuticals has been estimated as being currently over 40 billion dollars (approximately 30 billion euros) and estimates are that its growth rate may be more than 20% each year. The majority of this market is made up of protein biomolecules. This presents an ample opportunity and prospect for improving the quality of life of many people, in a significant manner. Unlike the traditional chemically synthesized pharmaceuticals, these protein biomolecules are isolated from a biological source and present new manufacturing challenges, especially in the interplay between bacterial expression and excretion, and the “downstream processing” to obtain the target substance from a mixture with other abundantly present, but undesired cells and substances. Since “down stream processing” can contribute 50-70% of the total cost of manufacturing a protein-based drug, efficient downstream processing to obtain the target biomolecule (the biopharmaceutical) is of substantial commercial importance. Although bacilli were considered as candidate organisms for the production of recombinant biopharmaceuticals by secretion before (Quax et al., 1993; Westers et al., 2004; Palva, 1982; Palva et al., 1983; Simonem et al., 1993; Udaka et al., 1993; Udaka, S., 1976; Ebisu et al., 1992; Miyauchi et al., 1999; Kajimo et al., 2000) successful secretion of human proteins from B. subtilis are scarcely reported, and when reported the secretion is inefficient or labile. Obstacles encountered include plasmid stability, proteolytic degradation of products and formation of intracellular inclusion bodies, among other impediments.

Poor yields in literature reports have prompted researchers to test several other organisms for secretion and production of recombinant hIL-3 (van Leen et al., 1991). However, problems such as insolubility (Davis et al., 1999) or degradation of produced hIL-3 (van Leen et al., 1991) have been reported.

Using secretion vectors in a proprietary Bacillus licheniformis host, active hIL-3 has been asserted to be purifiable in high yield from the growth medium without further need for refolding or modification (van Leen et al., 1991). However, it is noteworthy that the hIL-3 produced with B. licheniformis was engineered to lack four C-terminal residues, which are dispensable for full biological activity as seen from U.S. Pat. No. 5,516,512. The removal of these residues was said to preclude partial C-terminal cleavage of hIL-3 by unidentified proteases of B. licheniformis, but as can be seen from FIG. 3 of van Leen et al. (1991) residual proteolytic degradation still occurs. It is therefore reasonable to conclude that in order to improve the secretion and degradation of hIL-3 the poorly characterized host B. licheniformis is very unsuitable.

In view of the relatively long standing objective of obtaining biopharmaceuticals, especially recombinant biopharmaceuticals, from bacilli and the dearth of success with B. subtilus, it would be a significant advance in the art to achieve the secretion of correctly folded and fully biologically active cytokines like hIL-3 from bacilli, such as B. subtilis, especially in sufficiently increased and stable yield, that enables the formation of an integrated system with a subsequent downstream processing in a mild and highly natural manner.

### SUMMARY OF THE INVENTION

The foregoing and other objectives are achieved by the present invention. An aspect of the present invention encompasses a stable production system, which includes a method for producing cytokines and cytokine derived biomolecules in a maximal natural fashion, in which a target cytokine is efficiently produced by an expression system and secreted into a medium (growth medium etc.), in a stable form that is suitable for facile, mild and highly natural downstream processing.

Production rests in part on a recognition that the intrinsic properties of the cytokines that also enable their function in the human body. In general, the properties are two-fold. Under natural circumstances, cytokines are excreted by cells. In their natural state cytokines are at least a little hydrophobic and/or at least a little charged at certain places, which enables them to attach to their highly specific cellular docking sites (the cellular cytokine-receptors). In contrast cytokines are also not too hydrophilic as they would otherwise stick to any cell membrane and they are also not too heavily charged as they would be neutralized and to rapidly excreted by the human body mechanisms.

### BRIEF DESCRIPTION OF THE FIGURES

FIG. 1 is a schematic representation of hIL-3 over-production plasmids for B. subtilis.

FIG. 2 shows production of hIL-3 at different time points during growth.

FIG. 3 is a Western blot analysis of hIL-3 production by B. subtilis WB700.

FIG. 4 is a Western blot analysis of hIL-4 production by different protease-deficient strains.

FIG. 5 A is a mass spectrum of hIL-3 purified from a growth medium of B. subtilis WB700 p43LatlL3.

FIG. 5B is a mass spectrum of hIL-3 purified from a growth medium of B. licheniformis.

FIG. 6 shows AMS labelling of free thiol groups in purified hIL-3.

FIG. 7 graphically shows the bioactivity of hIL-3 produced by B. subtilis WB700.

### DETAILED DESCRIPTION OF THE INVENTION

An aspect of the present invention is a multi-stage method for expressing and recovering in higher concentration and in useful purities a cytokine, such as human Interleukin-3 (“hIL-3”).

In a first stage, the target is expressed in a microbial expression system, preferably bacterial, more preferably a Bacillus species, and most preferably a Bacillus subtilis strain, especially a strain from which at least 7 and preferably at least 8 extra-cellular proteases have been eliminated in addition to a significantly increased expression and processing and secretion. The removal of proteases enables convenient downstream processing under conditions more closely resembling natural circumstances.

In a further downstream processing stage, the cytokine is purified under mild conditions that are as natural as possible and in a facile purification procedure that also encompass mild materials. In this regard, the facile purification procedure may be a two-step process. Additional more particular features of this aspect of the present invention include the use of mild hydrophobic interaction chromatography in a first purification step to reduce the volume from the first purification step and the use of volatile buffers and salts that permit quick and convenient concentration by lyophilization and mass spectrometric analysis in any step.

The cytokine can be recovered and isolated as desired.

The present method enables the excretion of cytokines from bacteria (e.g., hIL-3) in concentrations suitable for scale-up to commercial production. For instance, up to 100 mg per litre in flask production and a well-adjusted, subsequent and convenient purification to greater than 95% purity (as measured by mass spectrometry) can, in principle, be achieved both on a lab and in pilot plant, and thus the present method is suitable for use in producing a target cytokine, such as hIL-3, on a commercial scale.

More particularly, the present invention relates to establishing a stable production system for cytokines, for instance hIL-3, based on a bacillus, such as a B. subtilis strain, and using the constitutively active P43 promoter in combination with a modified AmyL signal peptide (Lat, B. licheniformis a-amylase). The constitutively active P43 promoter is described in Wang (1984). An exemplary modified AmyL signal peptide is described in Quax et al. (1993), which may involve removing a so-called promiscuous second maturation site.

The Gram-positive bacterium Bacillus subtilis has the capacity to produce secreted bacterial enzymes. The advantages of using this Gram positive bacterium for recombinant protein expression, as compared to other micro-organisms, include the ability to secrete functional extra-cellular proteins directly into the culture medium, the lack of pathogenicity, and the absence of lipopolysaccharides (endotoxins) from the cell wall (Simonen, 1993). Nevertheless, as a general matter the secretion of pharmaceutically attractive recombinant proteins by this organism has heretofore frequently been found to be inefficient, and the lower concentration product is sufficiently impure as to present daunting challenges.

Accordingly, in an aspect of the present invention, the target cytokine is expressed in an expression system, most preferably a Bacillus subtilis strain from which at least 7 and preferably at least 8 extra-cellular proteases have been eliminated in addition to a significantly increased expression and processing and secretion to allow more facile downstream processing. As B. subtilis can secrete at least nine distinct proteases (Antelmann et al., 2001), which have the potential to degrade heterologous proteins (Westers et al., 2004), it is preferred that the strain herein be protease deficient. That is, for present purposes, a sufficiently protease deficient Bacillus subtilis strain can provide sufficient excretion, such as an at least 7, 8 or 9 protease deficient Bacillus subtilis strain, is preferred. For instance a protease deficient Bacillus subtilis strain, such as Bacillus subtilis WB800, can result in greater expression of the desired product. The B. subtilis WB800 strain is deficient in eight extra-cellular proteases for expression of heterologous genes, Wu et al. (1991). Strains that are deficient in 9 or more proteases can also be considered in principle for an expression system, e.g., as excretion cells, in another aspect of the present inventions.

Various strains are available to those skilled in the art, including the B. subtilis WB600 strain, the B. subtilis WB700 strain and B. subtilis WB800 strain, as examples.

Although the invention is not limited to the Bacillus subtilis species for the excretion, this bacterial strain is an example and is therefore described in more detail herein.

Various strains of bacteria and selected plasmids that were compared to the plasmid of our invention are presented in Table 1.

Subsequently the cytokine expressed and secreted is purified under continuing mild conditions that are as natural as possible (under non-denaturating conditions). For instance, the purification can be as facile as two purification steps that encompass mild materials. The choice of this approach is based on the above-described properties of cytokines in their natural state.

An aspect of this method is the use of mild hydrophobic interaction chromatography, for instance by using a weakly hydrophobic column, for instance by using a butyl group in the stationary phase, for instance using TSK-butyl as stationary phase, for instance (but not limited to) using Toyopearl Butyl-650C column material as stationary phase. These exemplary aspects of mild hydrophobic interaction are not intended to limit the scope of the present invention.

Another specific feature of this method is the use of mild ion exchange chromatography, for instance by using a weak ion exchange column, for instance by using an anion exchange material group in the stationary phase, for instance using Q-sepharose column as stationary phase, for instance under conditions of a low ionic strength. These exemplary aspects of mild ion exchange interaction are not intended to limit the scope of the present invention.

Other aspects of the present method include, for example, the use of mild methods to concentrate the proteins, for instance by a first step to reduce the volume in the first purification step the use of mild hydrophobic interaction chromatography. This can be accomplished, for instance, by using a weakly hydrophobic column, such as for instance using a butyl group in the stationary phase, for instance using TSK-butyl as stationary phase, for instance using Toyopearl Butyl-650C column material as stationary phase, which examples are non-limiting.

It can also been accomplished however by the use of volatile buffers and by the use of so-called volatile salts, such as for instance using ammonium bicarbonate or ammonium acetate or both, for instance but not limited to such salt and/or buffer being at a natural pH, such as using a pH of 7-7.5, such as using a pH of 7.2, which also enables quick and convenient concentration by lyophilization and mass spectrometric analysis in any step. These above described aspects of volatile buffers and the use of such so-called volatile salts are exemplary, and are not intended to limit the scope of the present invention.

The expressed cytokine that is extra-cellularly excreted from the expression system can be recovered, and, optionally, isolated in high yield and in stable form.

Isolated hIL-3 can exhibit bioactivity. This should be seen when in vitro testing using a suitable hIL-3 dependent cell line. Such cell lines are known and can be reasonably related to efficacy in vivo, such as in a patient. It will therefore be appreciated that pharmaceutical preparations containing the recovered and, optionally, isolated hIL-3 can be administered to effect such stimulation in a suitable cell line, and even in a mammal in need thereof. A mammal in need includes human.

It will be appreciated that techniques for purifying DNA as well as other techniques are known to those skilled in the art, as seen from Sambrook et al., 1989.

hIL-3 is a four-helix cytokine that can stimulate the proliferation and production of various blood cells. Since the present method can produce hIL-3 with the advantages described herein, it will be appreciated that the production of other human growth factors for instance, but not limited to other 4 helix-cytokines. In fact, there is no reason to assume any limitation on even to just other Interleukins (e.g. Interleukin 2 or Interleukin-5 which has the same cell receptor beta chain), since we have already also indication of successful production of TNF. Therefore molecules like GM-CSF (which has the same cell receptor beta chain) or TRAIL or FAS are in principle feasible too by adapting the principles of the present invention.

Last, but not least, it is conceivable that the excretion and subsequent natural downstream processing system renders problems for certain molecules. In addition, it is also conceivable that the molecules that cause this problem are also the molecules that cause problems in the human body in therapies. Consequently, it is also conceivable that this present highly natural integrated excretion and downstream processing system of the present invention could achieve a degree of mimicking of the human body and enable predictions of efficacy in animal tests or even clinical trials.

### EXAMPLES

Aspects of the present invention are described and illustrated in the following non-limiting Examples.

### Example 1

The bacterial strains and plasmids that are used are listed in Table 1. E. coli DH5alpha is used for construction of plasmids and is cultured in Luria Bertani broth (1.0% Bacto tryptone, 0.5% Bacto yeast extract and 0.5% NaCl). B. subtilis (Bacillus subtilis) strains are cultured in 2×TY, medium extra rich (MXR), or medium super rich (MSR). 2×TY containing 1.6% Bacto tryptone, 1.0% Bacto yeast extract, 1.0% NaCl, and 20 mM potassium phosphate buffer, pH 7.0. MXR medium that is used for over-expression of lipase (Lesuisse et al., 1993) contained 2.4% Bacto yeast extract, 1.2% casein hydrolysate, 0.4% Arabic gum, 0.4% glycerol, 0.17 M KH₂PO₄ and 0.72 M K₂HPO₄. MSR medium contains 2.5% Bacto yeast extract, 1.5% Bacto tryptone, 0.3% K₂HPO₄ and 1.0% glucose. If appropriate, trace elements are added from a 1000× stock solution (2 M MgCl₂, 0.7 M CaCl₂, 50 mMMnCl₂, 5 mMFeCl₃, 1 mMZnCl₂ and 2 mM thiamine). Antibiotics are used at the following concentrations: ampicillin (Ap), 100 microg/ml (E. coli); erythromycin (Em), 2.5 microg/ml (B. subtilis); hygromycin (Hyg), 100 microgml (B. subtilis); kanamycin (Km) 30 mg/ml (B. subtilis/E. coli).

Procedures for DNA purification, restriction, ligation, agarose gel electrophoresis and transformation of competent E. coli cells are known to those skilled in the art and exemplary procedures are carried out as described by Sambrook et al. (1989). Restriction endonucleases are obtainable from Invitrogen Life Technologies (UK), DNA polymerases are obtainable from Roche Diagnostics (Germany) and Stratagene (USA). The primers that are used for construction of the plasmids are from Invitrogen Life Technologies (UK) and are listed in Table 2. Amplified DNA fragments were purified with the Qiaquick PCR Purification Kit (Qiagen, Germany) or from gel using the Qiaquick Gel Extraction Kit (Qiagen, Germany).

Construction of the expression plasmids for production of hIL-3 is described.

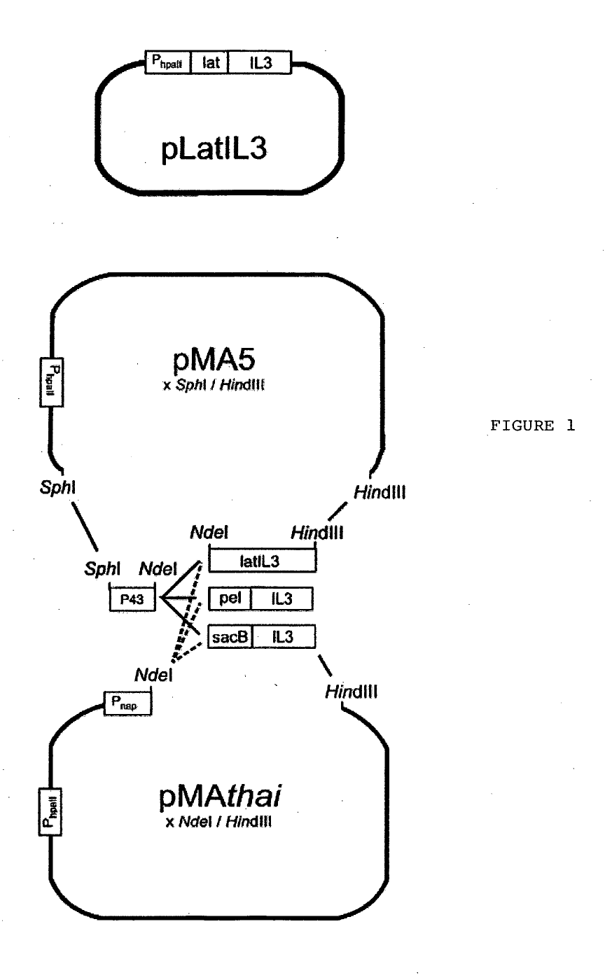

To investigate the expression and secretion of hIL3 by B. subtilis, the use of different promoters and signal sequences (FIG. 1) can be compared to the present invention. For this purpose, a series of plasmids are constructed based on the pUB 110 derived expression vector pMA5, which replicates in B. subtilis and E. coli (Bruckner et al., 1984; Zyprian, 1986; Dartois et al., 1994). For convenient cloning downstream the nap promoter sequence (Pnap) in the pMA5-derived plasmid pMAthai (Dröge et al., 2001), an NdeI site is introduced at the start codon of the nap gene via the so-called PCRbased QuikChange Site-Directed Mutagenesis method (Stratagene, USA) using the primers pMAthaiNdeIFor and pMAthaiNdeIRev. A second NdeI site present in the pMA series of plasmids is removed by the same method, using the primers deltaNdeIFor and deltaNdeIRev. For cloning of the P43 promoter, the upstream region of the cdd gene of B. subtilis 168, is amplified by PCR, using the primers P43ProF and P43ProR. A fragment containing the optimised B. licheniformis a-amylase signal sequence (amyL-SASA), followed by the hIL-3 gene is PCR-amplified from the pLatIL3 plasmid using the primers LatNdeIF and IL3R129. The B. subtilis signal sequences of pectate lyase (pel) and levansucrase (sacB) is PCR-amplifled from the B. subtilis 168 genone using the primers PelNdeIF and PeIIL3R, or SacBF and SacBIL3R, respectively. These two signal sequences are fused to the DNA sequence of the 129 amino acids variant of hIL-3 via the Splicing by Overlap Extension (SOE) method (Horton et al., 1989) after amplifying the hIL-3 part with the primers PeIIL3F or SacBIL3F and IL3R129. The resulting fragments latIL3, pelIL3 and sacBIL3 are cleaved with NdeI and HindIII and ligated into the NdeI and HindIII double digested pMAthai vector. Alternatively, these fragments are cleaved with NdeI, ligated to the NdeI digested P43 fragment, and are re-amplified by PCR with the P43ProF and IL3R129 primers. Subsequently, the resulting fragments are cleaved with SphI and HindIII and ligated into the SphI and HindIII double digested pMAS vector. Finally, the pBR322 origin of replication of the resulting plasmids is removed by BamHI digestion (pMAthai-derivatives) or SstI digestion (pMA5 derivatives). The subsequent self-ligation of the fragments with the promoter, signal sequence and hIL-3 combinations results in the pP43LatIL3 and in the plasmids pNapLatIL3, pNapPelIL3, pNapSacBIL3, pP43PelIL3 and pP43SacBIL3.

The method includes conditions to promote maximal production of hIL-3 consistent with parameters relating to secretion of bacterial enzymes. A selected B. subtilis strain can be transformed as described by Kunst and Rapoport (1995). For production of hIL-3, fresh transformants are preferably selected and grown in a suitable culture medium under suitable aeration, such as in a broth using broad-necked flasks filled to 20% of the flask volume for optimal aeration. The transformants are incubated, such as in flasks incubated at 37° C. for 24 hours under constant agitation (300 rpm). After 7 hours of growth in MSR medium, 0.1% citrate is added. The cells are removed by centrifugation for 30 min at 4500 rpm and the growth medium fractions are stored at −20° C.

A purification procedure is performed, such as described in PCT Int'l Publication WO 90/10705, which can be modified if desired. For hydrophobic interaction chromatography, solid NH₄Ac is added to growth medium fractions to a final concentration of 1.5 M (pH 7.2). Precipitated protein is removed by centrifugation for 15 min at 4500 rpm in 50 ml Greiner tubes. The supernatant is applied to a 20 ml Toyopearl Butyl-650C column (Supelco, USA), which is equilibrated with 50 mM (NH₄)HCO₃, 1.5 M NH₄Ac (pH 7.2) using a Duo Flow system (Bio-Rad, USA). After a washing step with 50 mM (NH₄)HCO₃, 1.5 M NH₄Ac (pH 7.2), hIL-3 is eluted in two peaks (A₂₈₀) during an isocratic flow of 50 mlvi (NH₄)HCO₃ (pH 7.3) and a following isocratic flow of demineralized water. A flow rate is 2 mlmin⁻¹ and the pressure is 47 psi during this procedure. The hIL-3 containing fractions are eluted at 50 mM (NR₄)HCO₃ were freeze-dried overnight, are dissolved in 5 mlvi (NH₄)HCO₃ (pH 7.8) and are applied to a 10 ml Q-Sepharose column (Amersham Pharmacia, USA). The hIL-3 fractions, which are obtained by elution with demineralized water are brought to pH 7.8 and are directly applied to a Q-Sepharose column. Finally, upon washing the Q-Sepharose column with 5 mM (NH₄)HCO₃ (pH 7.8), pure hIL-3 is collected from the flow through fraction. Protein concentration determinations are performed according to techniques known to those skilled in the art, such as those described in Bradford (1976).

Western blotting and immunodetection are performed. Samples are subjected to reducing SDS-PAGE according to Laemmli (1970), using 18% Ready Gels (Bio-Rad, USA). Alternatively, 4-12 or 12% NuPage Novex Bis-Tris Gels in combination with, respectively, MES or MOPS SDS Running Buffer (Invitrogen Life Technologies, USA) is used. The Low-Range Rainbow™ Molecular Weight Marker (Amersham Pharmacia, USA) or SeeBlue Plus2 Pre-Stained Standard (Invitrogen Life Technologies, USA) is used to determine the apparent molecular weight of separated proteins. After electrophoresis the gels are stained with Coomassie Brilliant Blue (Neuhoff et al., 1988) or blotted to a Protran mitrocellulose transfer membrane (Schleicher and Schuell, USA) as described by Kyhse-Andersen (1984), for example. For a dot blot, 10 microliter samples of culture supernatant are spotted on the membrane, after which the membrane is air-dried. Membranes are blocked for 1 hour or overnight using 5% fat-free milk (Nutricia, The Netherlands), 0.05% Tween 20, 150 mM NaCl, 10 mM Tris-HCl pH 8.0 (MTBST), then are incubated overnight with a 1:5000 dilution of a rabbit polyclomal anti-hIL3 antibody (Sigma-Aldrich, USA) in MTBST. After washing in 0.05% Tween 20, 150 mM NaCl, 10 mM Tris-HCl pH 8.0 (TBST), membranes are incubated for 1 h with alkaline phosphatase or horseradish peroxidase-conjugated goat anti-rabbit IgG secondary antibody (Biosource, USA) diluted 1:10,000 in TBST. Finally, the presence of hIL-3 is visualized using alkaline phosphatase detection or chemoluminescent detection as is know to those skilled in the art, such as described in Sambrook et al., 1989.

Detection of free thiol groups in hIL-3 is next described. Samples of 10 microM hIL-3 are incubated in the presence or absence of a 10-fold molar excess of dithiothreitol (DTT), after which 25 mM 4-acetamido-4′-maleimidylstilbene-2,2′-disulfonic acid (AMS) (Molecular Probes, USA) is used for crosslinking to free thiol groups (30 min incubation at 37° C.). For subsequent visualization of AMS crosslinked to hIL-3 by SDS-PAGE, a non-reducing sample buffer is used.

Analysis of purified hIL-3 using MALDI-TOF is next described. Samples of 30 microM hIL-3 in 5 mM (NH₄)HCO₃ (pH 7.8) are diluted three times in 0.1% TFA and are mixed 1:1 with matrix solution consisting of 20 mg/ml alpha-cyano-4-hydroxy cinnamic acid (Sigma-Aldrich, USA) in a 70/30 (v/v) solution of acetonitril and 0.1% TFA. A reference protein set containing cytochrone c (12,361 Da) and myoglobin (16,952 Da) in the same matrix is used following the same procedure. Besides the singly charged ions of both reference proteins, the doubly charged ion of cytochrone c (at m/z 6181) is used for calibration. Spectra are recorded on a TofSpec-E MALDI mass spectroneter (Micromass Inc., UK).

Molecular weight is described as average molecular mass unless stated otherwise herein. The bioactivity of purified hIL-3 is determined by measuring the hIL-3-dependent stimulation of the ³H-thymidime (³H-TdR) uptake by the human leukaemia cell line M07e. Cells (50×10³) are cultured in 150 microliter RPMI 1640 medium with 100 U penicillin, 100 U streptomycin, and 1% FBS (Foetal Bovine Serum). The cells are incubated with or without hIL-3 in a 96-well round-bottomed microtiter plate in triplicate. After 4 days of culturing (37° C. and 5% C0₂), 6 h prior to cell harvest, 0.1 microCi ³H-TdR with a specific activity of 2 Ci/mmol are added to each well. Radioactivity uptake is determined by liquid scintillation counting. Values are expressed as disintegrations per second (DPS).

In an aspect of the present invention, the expression of hIL-3 in B. subtilis can be maximized by selecting appropriate conditions. Conditions suited to being scalable to commercial scale are presently preferred.

A first approach towards this objective in the hIL-3 production, B. subtilis DB 104 is transformed with pLatIL3 and production of hIL3 by cells grown in 2×TY, 1×MXR or 1×MSR medium are followed in time. Upon growth in 1×MXR the highest optical densities (OD₆₀₀ of 12) are obtained. Nevertheless, the highest yields of extracellular protein (400 mg/l) are obtained upon growth in 1×MSR. Even under these optimized conditions for growth and protein secretion, the production of hIL-3 was below 100 microgram/l.

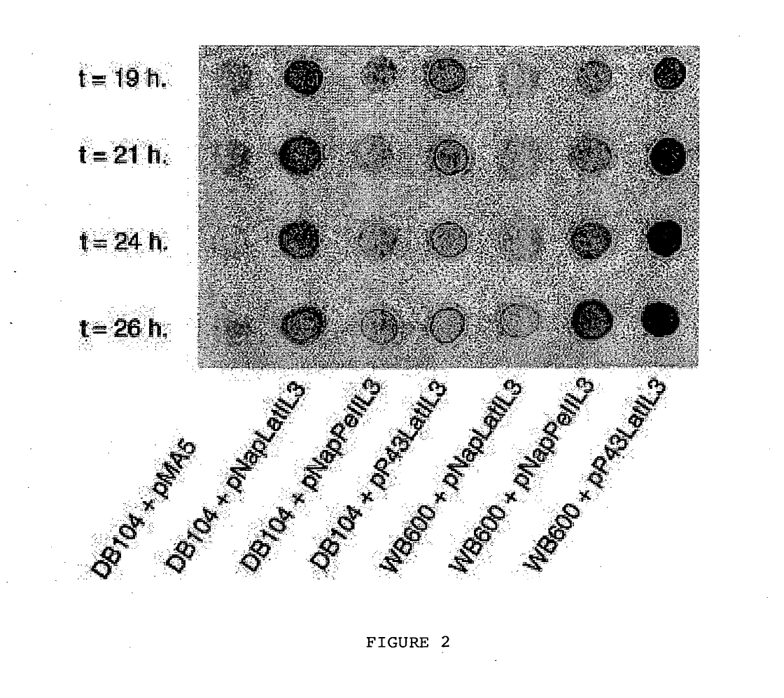

Since this production level is not commercially relevant, two series of vectors can be constructed for optimalization of hIL-3 production: besides the pP43LatIL3, the pP43Pel1L3 and pP43SacBIL3 series and the pNapLatIL3, pNapPel1L3 and pNapSacBIL3 series. The pNap plasmids carry signal sequence-hIL-3 fusions under the transcriptional control of the strong nap promoter and the pP43 plasmids carry these fusions under the control of the strong P43 promoter. These plasmids are used to transform B. subtilis strain DB 104 or the six-fold protease-deficient strain B. subtilis WB600. Next, the production of hIL-3 is studied at different time intervals of growth on 1×MSR medium. Dot blot analysis with specific hIL-3 antibodies revealed that the highest levels of extra-cellular hIL-3 were obtained with B. subtilis WB600 containing the pP43LatIL3 vector (FIG. 2). Furthermore, the highest yield of hIL-3 produced by this strain was observed after 24-26 hours of growth. Western blot analysis shows that substantial amounts of the extracellular hIL-3 are subject to partial proteolysis, even when this protein was produced in B. subtilis WB600.

To further establish preferred the growth conditions for the production of hIL-3 by B. subtilis WB600 pP43LatIL3, media with different combinations of carbon sources are tested. These media can contain 1% xylose, lactose or arabinose in combination with 0.1% glucose, or 1% glucose only. Dot blot analysis reveals that the use of MSR medium containing 1% xylose plus 0.1% glucose results in about three-fold higher levels of secreted hIL-3 than MSR with 1% glucose. Therefore, MSR medium with 1% xylose and 0.1% glucose are used in all further experiments. In addition, this medium is supplemented with trace amounts of metal ions, because such ions may act as folding catalysts for certain secreted proteins (Sarvas et al., 2004). Although not systematically tested, the presence of metal ions is presently expected to result in a slightly improved hIL-3 production.

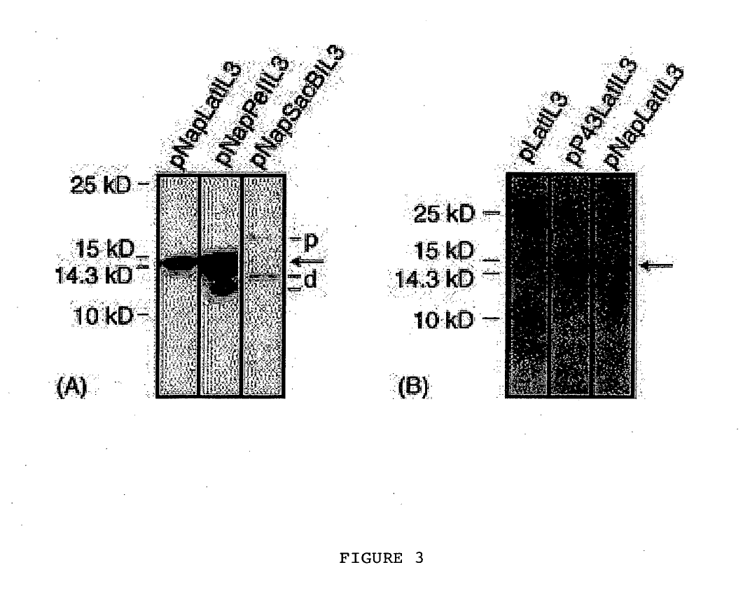

To re-evaluate hIL-3 production with the help of the different hIL-3 expression plasmids under optimized conditions of growth, the B. subtilis strain WB700 is used. This strain has the same protease mutations as B. subtilis WB600 but, in addition, it lacks the extracellular Vpr protease. To assess hIL-3 production by the B. subtilis WB700 strain, fresh transformants are grown for 24 hours. Next, cells and growth medium are separated by centrifugation, and the levels of secreted hIL-3 are compared by Western blotting (FIG. 3). The highest levels of hIL-3 secretion are reached when the nap promoter is used in combination with the Pel signal peptide. However, plasmids encoding these promoter-signal peptide combinations appear to be structurally unstable. Also, a large moiety of the secreted hIL-3 is partially degraded (FIG. 3A), which is regarded as a major disadvantage for subsequent purification. Second highest is the combination of the P43 promoter and the lat signal sequence. Since this particular promoter-signal sequence combination neither results in structural plasmid instability nor the appearance of hIL-3 degradation products (FIG. 3B), pP43LatIL3 is considered to be more robust. Importantly, very little, if any, hIL-3 is detectable by Western blot analysis of the intracellular fraction of cells containing pP43LatIL3, indicating efficient intracellular processing of the Lat-hIL-3 precursor and subsequent secretion of the mature protein. This is not the case for all constructs that may be tested. For example, the SacB-hIL-3 precursor is not efficiently processed and, in fact, this precursor was even detectable in the growth medium of cells containing pNapSacBIL3, which may occur due to lysis of cells (FIG. 3A).

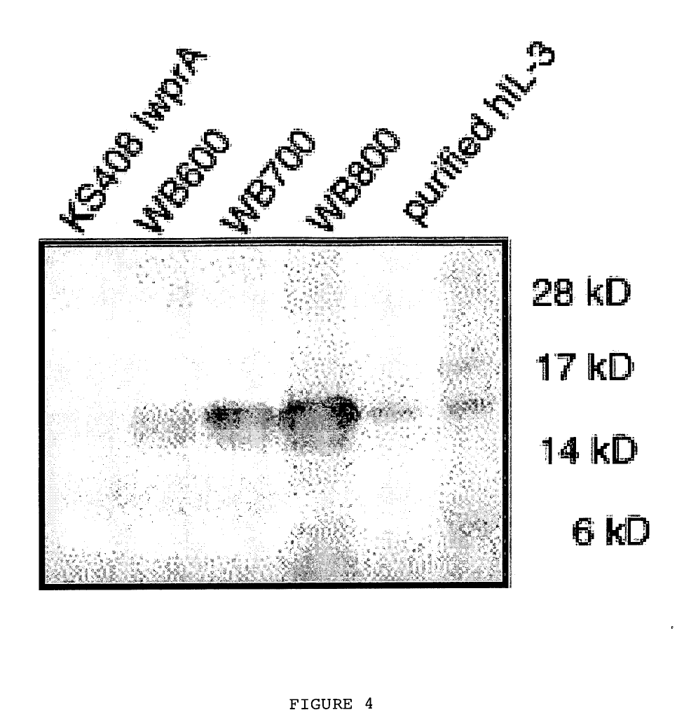

For the purposes of the present invention, it is desired to provide conditions conducive to maximize production to a target cytokine that can be facilely and economically purified to near homogenity. This can be achieved as seen from studying and testing the influence of the wall protease A on the production of hIL-3 using B. subtilis WB800. This strain has an inactivated wprA gene and lacks the proteases that are also absent from B. subtilis the WB700 strain. As shown in FIG. 4, the use of B. subtilis WB800 in combination with the pP43LatIL3 plasmid results in significantly elevated levels of hIL-3 production, as compared to the B. subtilis WB600 and B. subtilis WB700 strains containing the same plasmid. As may be seen by densitonetric analyses of Coomassie Brilliant Blue stained SDS-PAA gels, B. subtilis WB800 pP43LatIL3 may produce at least about 100 mg of hIL-3 per litre. The absence of WprA alone from B. subtilis may not be sufficient to obtain hIL-3 production levels that are detectable by Western blotting, as seen with the wprA mutant B. subtilis strain K5408 IwprA (FIG. 4).

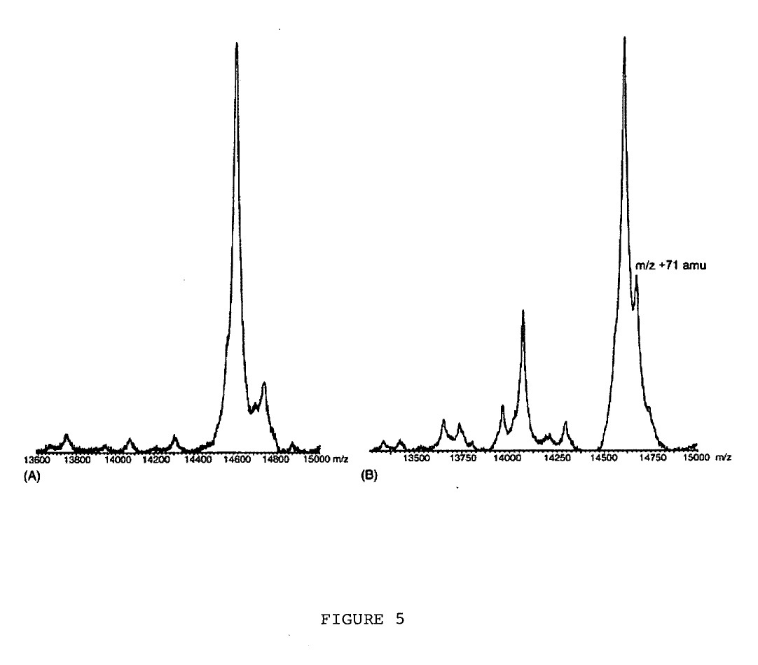

Exemplary purification and analyses of hlL-3 produced in B. subtilis WB700 is described. It will be appreciated that the secreted hIL-3 from the culture broth of B. subtilis WB700 containing the pP43LatIL3 construct can be purified to almost homogeneity. For instance, after a first chromatographic step (such as hydrophobic interaction chromatography) two elution peaks (A₂₈₀) containing hIL-3 are detectable: one at the end of the first gradient (50 mM NH₄Ac) and a second during elution with demineralised water. Biochemically the material from both peaks is in distinguishable. The protein fractions, which emerge during elution with demineralized water, are directly applied to a Q-Sepharose column. The peak fraction at 50 mM NH₄Ac is freeze-dried and dissolved in 5 mM (NH₄)HCO₃, before it is applied to the Q-Sepharose column. As may be shown by SDS-PAGE and Western blotting, fractions that are obtained from Q-Sepharose chromatography of the samples derived from the first chromato-graphic step contain hIL-3-specific protein bands in the range of about 13-14.5 kDa. The mass spectrum of the purified hIL-3 shows a prominent peak at m/z 14606.1 (FIG. 5A), which is consistent with the mass spectrum observed for hIL-3 that has been reportedly produced in B. licheniformis (m/z 14594.6) (van Leen et al., 1991) and that can be used as a reference for present purposes (FIG. 5B). Notably, the reference material reveals am additional peak at m/z 14665.4, which relates to an alternative maturation site in the AmyL signal peptide that is absent from the Lat signal peptide. Maturation at the second site results in the presence of an additional N-terminal alanine residue in hIL-3, which can explain the 71 Da increment (Bonekamp et al., 1998). Besides these main peaks, some fractions contain products with masses of about m/z 14,100 and m/z 13,700. The smallest fragment can be analyzed by N-terminal amino acid sequencing.

The results show that this fragment corresponds with an N-terminal hIL-3 degradation product of 120 amino acid residues starting at Lys 10. Consistent with the mass spectrometric analysis, the theoretical mass of this degradation product is 13705.7 Da.

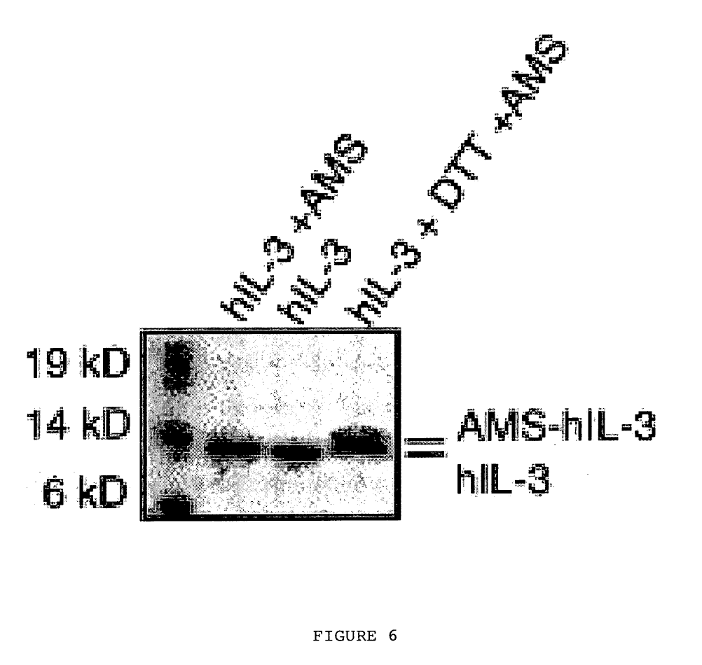

hIL-3 is known to contain one intra-molecular disulfide bond. To verify the correct formation of this bond, AMS labeling experiments can be performed. AMS will only cross-react with free thiol groups, thereby causing a reduced mobility of a cross-linked protein in SDS-PAGE. As shown by SDS-PAGE, AMS does not cross-react with a hIL-3 purified from the growth medium of B. subtilis. In fact, incubation of hIL-3 with AMS results in a reduced mobility of SDS-PAGE only when hIL-3 is reduced with DTT prior to the incubation with AMS. This implies that there are no free thiol groups present in the purified protein (FIG. 6). Since hIL-3 contains only two cysteine residues, the lack of AMS cross-linking is an indication that the disulfide bond in hIL-3 is properly formed by B. subtilis.

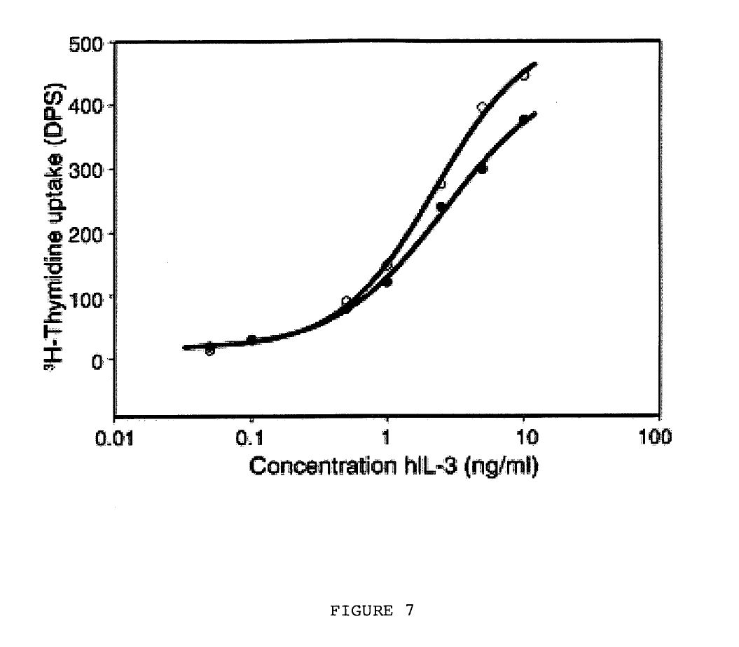

Bioactivity of hIL-3 (purified) from a growth medium of B. subtilis WB700 can be tested using the hIL3-depemdent leukaemia cell line M07e and a thymidine uptake assay. As shown in FIG. 7, the thymidine uptake curve displayed by cells that are stimulated with hIL3 produced in B. subtilis compare very well with the curve displayed by cells that are stimulated with hIL3 that was previously purified from B. licheniformis. This B. licheniformis-produced hIL-3 is known to have full biological activity (van Leen et al., 1991). Additionally, the thymidime uptake that may be observed upon incubation of M07e cells with 10 mg/mlhIL-3 purified from E. coli is in the 450-500 DPS range. The hIL-3 that can be recovered from each of the two peaks eluted after the first chromatographic purification step can display comparable bioactivity. This shows that the present system for high-level production of properly folded and bioactive hIL-3 is established on the basis of protease-mutant strains of B. subtilis.

In order to facilitate product recovery and to avoid a cell breakage step, secretion of hIL-3 in the growth medium by linking a signal Sequence to the hIL-3 gene can be applied. When the modified AmyL signal peptide (Lat) is used, a reproducibly high secretion of hIL-3 from the cells may be obtained. The signal peptide is removed correctly during secretion as demonstrated by mass spectrometric analysis. This is in contrast to the 20% miscleavage of the authentic AmyL signal peptide from hIL-3 precursor in B. licheniformis (Bonekamp et al., 1998). The SacB signal peptide did not result in productive secretion of hIL-3, although efficient secretion of staphylokinase under guidance of this signal peptide has been reported (Ye et al., 1999). The Pel signal peptide incidentally gives rise to a high-level of secreted product in the medium. However, the plasmid instability that is observed when the pel signal sequence is used in combination with the nap promoter suggests that high-level production of the Pel-hIL-3 precursor may be detrimental for the cells. This can result in a selective growth advantage of cells that have lost the ability to produce this precursor. Additionally, the occurrence of a corresponding degradation product may be an indication that the production of Pel-hIL-3 at high-levels elicits a secretion stress response, as previously observed upon high-level production of Bacillus alpha-amylases (Hyyrylainen et al., 2000 and Darmom et al., 2002). Such a secretion stress response would result in the production of HtrA-like proteases at elevated levels and, in turn, this could result in increased product degradation.

In the present invention, the use of B. subtilis strains with increasing numbers of mutated protease genes can result in a stepwise improvement of hIL-3 accumulation in the medium. Even after purification of hIL-3 from the culture medium of the B. subtilis WB700 strain and analyses of the fractions, some degradation products may still be detectable. N-terminal sequencing of the smallest degradation product that can be detected by MALDI-TOF in some fractions reveals that degradation may take place at B. subtilis the N-terminus of the protein. A major increase in hIL-3 level is observed upon using B. subtilis WB800, which lacks WprA, a cell wall protease implicated in the degradation of slowly folding proteins in the cell wall. However, WprA is not the only protease degrading hIL-3 as can be inferred from the absence of hIL-3 in the supernatant of strain K5408 IwprA, which lacks only WprA. It may therefore be considered that a cytokine like hIL-3 is always prone to proteolysis by multiple cell wall-proteases and extracellular proteases of B. subtilis. In such a case, in principle, the proteolysis of exported hIL-3 molecules can occur at all stages of the secretion process, starting immediately after translocation across the membrane and continuing in the cell wall environment and growth medium.

Furthermore, it is considered that hIL-3 produced by an organism, such as B. subtilis, according to the present method is properly folded. This may be shown if the two cysteine residues in hIL-3 produced by B. subtilis are oxidized, which is most likely due to the correct formation of an imtra-molecular disulfide bond. It is not known how this disulfide bond is formed. Although not wishing to be bound by a particular theory, it is conceivable that the BdbCD system may be involved in that aspect of the process. Both BdbC and BdbD are required for the folding of exported proteins containing disulfide bonds (Bolhuis et al., 1999; Meima et al., 2002).

When appropriate adaptations to the expression and secretion signals used are applied, as seem with the present invention, a convenient and practically feasible production of cytokines like hIL-3 is achievable. For a larger scale production of hIL-3, a preferred production system both with regard to stability and yield is preferably predicated on a sufficiently protease deficient strain of bacteria, such as a modified B. subtilis WB800 strain in combination with the pP43LatIL-3 vector, including the modified AmyL signal sequence. After 24 hours of culturing, this strain can yield at least about 100 mg per litre hIL-3, which should be sufficient to support scale-up to a commercial production process.

In view of the production of hIL-3, the present system should be applicable for the production of other heterologous proteins, for instance, but not limited to four-helix bundle cytokines related to hIL-3, that do not require glycosylation to achieve full biological activity.

The complete description (disclosure) of each literature reference, patent document or publication identified in this application is incorporated herein by reference (including such references as may be cited within any such literature or patent documents or publications), and these include: Antelmann, H., et al., 2001, A proteomic view on genome-based signal peptide predictions, GenomeRes. 11, 1484-1502; Avanzi, G. C. et al., 1990, M-07e human leukemic factor dependent cell line provides a rapid and sensitive bioassay for the human cytokines GM-CSF and IL-3, J. Cell Physiol. 145, 458-464; Bolhuis, A., et al., 1999, Functional analysis of paralogous thiol-disulfide oxidoreductases in Bacillus subtilis, J. Biol. Chem. 274, 24531-24538; U.S. Pat. No. 5,705,362; Bradford, M M., 1976, A rapid and sensitive method for the quantitation of microgram quantities of protein utilizing the principle of protein-dye binding. Anal. Biochem., 72, 248-254; Bruckner, R., et al., 1984, Expression of a chloramphenicol-resistance determinant carried on hybrid plasmids in gram-positive and gram-negative bacteria, Gene 32, 151-160; Darmon, E., et al., 2002, A novel class of heat and secretion stress-responsive genes is controlled by the autoregulated CssRS two-component system of Bacillus subtilis, J. Bacteriol. 184, 5661-5671; Dartois, V., et al., 1994, Genetic analysis and overexpression of lipolytic activity in Bacillus subtilis. Appl. Environ. Microbiol., 60, 1670-1673; Davis, G. D., et al., 1999, New fusion protein systems designed to give soluble expression in Escherichia coli. Biotechnol. Bioeng., 65, 382-388; Dorssers, L., et al., 1987, Characterization of a human multilineage-colony-stimulating factor cDNA clone identified by a conserved noncoding sequence in mouse interleukin-3, Gene 55, 115-124; U.S. Pat. No. 5,516,512 (Dorssers et al. 1996); Dröge, M. J., et al., 2001, Paralogous gene analysis reveals a highly enantioselective 1,2-O-isopropylideneglycerol caprylate esterase of Bacillus subtilis, Eur. J. Biochem. 268, 3332-3338; Ebisu, S., et al., 1992, Production of human epidermal growth factor by Bacillus brevis increased with use of a stable plasmid from B. brevis 481, Biosci. Biotechnol. Biochem. 56, 812-813; Ebisu, S., et al., 1996, The efficient production of human epidermal growth factor by Bacillus brevis, Ann. N.Y. Acad. Sci. 782, 115-122; Eder, M., et al., 1997, IL-3 in the clinic, Stem Cells 15, 327-333; Harwood, C. R., 1992. Bacillus subtilis and its relatives: molecular biological and industrial workhorses, Trends Biotechnol. 10, 247-256; Horton, R. M., et al., 1989, Engineering hybrid genes without the use of restriction enzymes: gene splicing by overlap extension, Gene 77, 61-68; Hyyryffiinen, H. L., et al., 2000, D-Alanine substitution of teichoic acids as a modulator of protein folding and stability at the cytoplasmic membranel/cell wall interface of Bacillus subtilis, J. Biol. Chem. 275, 26696-26703; Kajino, T., et al., 2000, A protein disulfide isomerase gene fusion expression system that increases the extracellular productivity of Bacillus brevis, Appl. Environ. Microbiol. 66, 638-642; Kawamura, F., Doi, R. H., 1984, Construction of a Bacillus subtilis double mutant deficient in extracellular alkaline and neutral proteases. J. Bacteriol. 160, 442-444; Kobayashi, K. et al., 2003, Essential Bacillus subtilis genes, Proc. Natl. Acad. Sci. U.S.A. 100, 4678-4683; Kunst, E, Rapoport, G., 1995, Salt stress is an environmental signal affecting degradative enzyme synthesis in Bacillus subtili, J. Bacteriol. 177, 2403-2407; Kunst, F., et al., 1997, The complete genone sequence of the gram positive bacterium Bacillus subtilis. Nature 390, 249-256; Kyhse-Andersen, J., 1984, Electroblotting of multiple gels: a simple apparatus without buffer tank for rapid transfer of proteins from polyacrylamide to nitrocellulose, J. Biochem. Biophys. Methods 10, 203-209; Laemmli, U. K., 1970, Cleavage of structural proteins during the assembly of the head of bacteriophage T4, Nature 227, 680-685. Lesuisse, E., et al., 1993, Purification and preliminary characterization of the extracellular lipase of Bacillus subtilis 168, an extremely basic pH-tolerant enzyme, Eur. J. Biochem. 216, 155-160; Mangi, M. H., et al., 1999, Interleukin-3 in hematology and oncology: current state of knowledge and future directions, Cytokines Cell. Mol. Ther. 5, 87-95; Meima, R., et al., 2002, The bdbDC operon of Bacillus subtilis encodes thiol-disulfide oxidoreductases required for competence development, J. Biol. Chem. 277, 6994-7001; Miyauchi, A., et al., 1999, Structural conversion from non-native to native form of recombinant human epidermal growth factor by Brevibacillus choshinensis, Biosci. Biotechnol. Biochem. 63, 1965-1969; Murashima, K., et al., 2002, Heterologous production of Clostridium cellulovorans engB, using protease-deficient Bacillus subtilis, and preparation of active recombinant cellulosomes, J. Bacteriol. 184, 76-81; Neuhoif, V., et al., 1988, Improved staining of proteins in polyacrylamide gels including isoelectric focusing gels with clear background at nanogram sensitivity using Coomassie Brilliant Blue G-250 and R-250, Electrophoresis 9, 255-262; Norrander, J., et al., 1983, Construction of improved MI 3 vectors using oligodeoxynucleotide-directed mutagenesis, Gene 26, 101-106; Palva, I., 1982, Molecular cloning of alpha-amylase gene from Bacillus amyloliquefaciens and its expression in B. subtilis, Gene 19, 81-87; Palva, I., et al. 1983, Secretion of interferon by Bacillus subtilis. Gene 22, 229-235; Park, S., et al., 1988, Modulation of folding pathways of exported proteins by the leader sequence, Science 239, 1033-1035; PCT Int'l Patent Publication WO 90110705; Quax, W. J. et al., 1993, Correct secretion of heterologous proteins from Bacillus licheniform, in Baltz, R. H. et al., (Eds.), Industrial Microorganisms: Basic an Applied Molecular Genetics. ASM, Washington, pp. 143-150; Sambrook, J., et al., 1989, MolecularCloning: A Laboratory Manual, second ed. Cold Spring Harbor Laboratories, Cold Spring Harbor, N.Y.; Sarvas, M., et al., 2004, Post-translocational folding of secretory proteins in Gram-positive bacteria, Biochim. Biophys. Acta 1694, 311-327; Simonen, M., et al., 1993, Protein secretion in Bacillus species, Microbiol. Rev. 57, 109-137; Stephenson, K., et al., 1998, Influence of a cell-wall associated protease on production of alpha-amylase by Bacillus subtilis, Appl. Environ. Microbiol. 64, 2875-2881; Tjalsma, H., et al., 2000, Signal peptide-dependent protein transport in Bacillus subtilis: a genone-based survey of the secretone, Microbiol. Mol. Biol. Rev. 64, 515-547; Tjalsma, H., et al., 2004, Proteomics of protein secretion by Bacillus suhtilis: separating the “secrets” of the secretone, Microbiol. Mol. Biol. Rev. 68, 207-233; Udaka, S., 1976, Screening for protein-producing bacteria, Agric. Biol. Chem. 40, 523-528; Udaka, S., et al., 1993, High-level secretion of heterologous proteins by Bacillus brevis, Methods Enzymol. 217, 23-33; van Leen, R. W., et al., 1991, Production of human interleukin-3 using industrial microorganisms. Biotechnology (N.Y.) 9, 47-52; Wang, P. Z et al., 1984, Overlapping promoters transcribed by Bacillus subtilis sigma 55 and sigma 37RNA polymerase holoenzymes during growth and stationary phases. J. Biol. Chem. 259, 8619-8625; Westers, L., et al., 2004, Bacillus subtilis as cell factory for pharmaceutical proteins: a biotechnological approach to optimize the host organism, Biochim. Biophys. Acta 1694, 299-310; Wu, X. C., et al., 1991, Engineering a Bacillus subtilis expression-secretion system with a strain deficient in six extracellular proteases, J. Bacteriol. 173, 4952-4958; .-C. Wu, J. C. et al., 2000, presentation at the 11th International Conference of Antibody Engineering, San Diego, Calif.; Yang, Y. C., et al., 1986, Human IL-3 (multi-CSF): identification by expression cloning of a novel hematopoietic growth factor related to murine IL-3, Cell 47, 3-10; Ye, R., et al., 1999, High-level secretory production of intact, biologically active staphylokinase from Bacillus subtilis, Biotechnol. Bioeng. 62, 87-96; Zenke, G., 1991, Purification and characterization of natural human interleukin-3, Lymphokine Cytokine Res. 10, 329-335; Zyprian, E., et al, 1986, Characterization of signals promoting gene expression on the Staphylococcus aureus plasmid pUBI 10 and development of a gram-positive expression vector system, DNA 5, 219-225.

TABLE 1
Strains and plasmids

| Strains | Genotype/relevant properties^{a} | Source/reference |
|---|---|---|
| E. coli | | |
| DH5α | F⁻ Φ80dlacZΔM15 endA1 recA1 gyrA96 thi-1 hsdR17(r_{K}⁻ m_{K}⁻) supE44 | Invitrogen Life |
| | relA1 deoR Δ(lacZYA-argF) U169 | Technologies (USA) |
| Top10F′ | F′ {lacI^{q} Tn10(Tet^{R})} mcrA Δ(mrr-hsdRMS-mcrBC) Φ80lacZΔM15 | Invitrogen Life |
| | ΔlacX74 deoR recA1 araD139 Δ(ara-leu) 7697 galU galK rpsL endA1 nupG | Technologies (USA) |
| B. subtilis | | |
| 168 | trpC2 | Kunst et al. (1997) |
| DB104 | his nprE aprE | Kawamura and Doi (1984) |
| WB600 | trpC2 nprE aprE epr bpr mpr nprB; Em^{r} | Wu et al. (1991) |
| WB700 | trpC2 nprE aprE epr bpr mpr nprB vpr; Em^{r} | Ye et al. (1999) |
| WB800 | trpC2 nprE aprE epr bpr mpr nprB vpr wprA; Hyg^{r} | Murashima et al. (2002) |
| KS408 IwprA | trpC2 amyE; xylose-inducible amyL; wprA::pMutin2; Em^{r} Cm^{r} | Stephenson and Harwood |
| | | (1998) |
| Plasmids | Relevant properties^{a} | Source/reference |
| pUC18 | P_{lac}, ColE1, Ap^{r} | Norrander et al. |
| | | (1983) |
| pLatIL3 | Vector for production and secretion of hIL-3 by means of a modified AmyL | Quax et al. (1993); |
| | signal peptide; Km^{r} | Dorssers and van |
| | | Leen (1996) |
| pMA5 | pUB110 derivative, ColE1, repB, Km^{r}, Ap^{r}, P_{hpaII} | Dartois et al. (1994) |
| pMAthai | pMA5 derivative, containing the B. subtilis Thai 1-8 nap gene, downstream of | Dröge et al. |
| | the HpaII and nap promoter | (2001) |
| pNapLatIL3 | pMAthai derivative, containing the hIL-3 gene with the lat signal sequence, | This study |
| | downstream of the HpaII and nap promoter | |
| pNapPelIL3 | pMAthai derivative, containing the hIL-3 gene with the pel signal sequence, | This study |
| | downstream of the HpaII and nap promoter | |
| pNapSacBIL3 | pMAthai derivative, containing the hIL-3 gene with the sacB signal sequence, | This study |
| | downstream of the HpaII and nap promoter | |
| pP43LatIL3 | pMA5 derivative, containing the hIL-3 gene with the lat signal sequence, | This study |
| | downstream of the HpaII and P43 promoter | |
| pP43PelIL3 | pMA5 derivative, containing the hIL-3 gene with the pel signal sequence, | This study |
| | downstream of the HpaII and P43 promoter | |
| pP43SacBIL3 | pMA5 derivative, containing the hIL-3 gene with the sacB signal sequence, | This study |
| | downstream of the HpaII and P43 promoter | |

^{a}Km^{r}, kanamycin resistance marker; Em^{r}, erythromycin resistance marker; Ap^{r}, ampicillin resistance marker; Hyg^{r}, hygromycin resistance marker.

TABLE 2

| Primers^{a} 5′ → 3′ | |
|---|---|
| pMAthaiNdeFor | GGGAGGGGCATTCATATGTCAAACCATTC^{a} |
| pMAthaiNdeRev | GAATGGTTTGACATATGAATGCCCCTCCC |
| Δ NdeFor | GGAGCGATTTACACATGAGTTATGCAG |
| Δ NdeRev | CTGCATAACTCATGTGTAAATCGCTCC |
| P43ProF | GGGCGCATGCACTTTTAAATACAGCCATTG |
| P43ProR | CCGCCCATATGTACATTCCTCTCTTACC |
| latNdeIF | GGGAGGAGACATATGAAACAACAAAAACGG |
| IL3R129 | CCACCCCAAGCTTCTAGCTCAAAGTCG |
| pelNdeIF | GCCCGGCCATATGAAAAAAGTGATGTTAG |
| pelIL3R | CATGGGAGCTGCGTTCGCGCCTGCTGGAG |
| pelIL3F | CGAACGCAGCTCCCATGACCCAGACAACG |
| sacBF | GGGGTATACAGCATATGAACATCAAAAAG |
| sacBIL3R | CATGGGAGCCGCAAACGCTTGAGTTG |
| sacBIL3F | GCGTTTGCGGCTCCCATGACCCAGAC |

^{a}Introduced restriction sites are depicted in bold. point mutations are underlined.

What I claim is:

1. A production method comprising expressing a mammalian protein in a bacterium that does not excrete toxic compounds, and wherein the protein is excreted in a medium, and purifying the excreted protein by at least one subsequent purification step, wherein the purification does not involve denaturation of the protein.

2. A method according to claim 1 wherein the protein is a human protein.

3. A method according to claim 1 wherein the protein is a human cytokine.

4. A method according to claim 1 wherein the purification steps include only mild interaction with a stationary phase.

5. A method according to claim 4 wherein the stationary phase is a mildly hydrophobic substance or a weak anionic or a weak cationic exchange substance.

6. A method according to claim 1 wherein the method further comprises concentrating the protein by on-column or by freeze drying.

7. A method according to claim 6 wherein in the method buffer-systems and salts are present and are volatile under conditions of freeze drying.

8. A method according to claim 1 wherein the bacterium is a GRAS organism.

9. A method according to claim 1 wherein the bacterium is a protease deficient bacterium.

10. A method according to claim 1 wherein the bacterium is a protease deficient bacterium that is capable of a normal rate of growth and that retains the capability of excreting the target protein in normal amounts.

11. A method according to claim 1 wherein the bacterium is a protease deficient bacterium with at least 7 proteases knocked out.

12. A method according to claim 1 wherein the bacterium is a protease deficient bacterium with at least 8 proteases knocked out.

13. A method according to claim 1 wherein the bacterium is selected from the species Bacillus.

14. A method according to claim 1 wherein the bacterium is Bacillus subtilis.
